(19) United States
(12) Patent Application Publication
(10) Pub. No.: US 2009/0123988 A1
(43) Pub. Date: May 14, 2009

(54) METHOD FOR THE SPECIFIC OR NON-SPECIFIC SEPARATION OF CELLS AND/OR VIRUSES FROM LIQUID MEDIA AND THE USE THEREOF

(75) Inventors: Andreas Holländer, Potsdam (DE); Michael Keusgen, Marburg (DE); Johannes Kramer, Swisttal (DE); Ansgar Ferner, Montabaur (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: Hansastrasse 27 c,, Munchen (DE); Fraunhofer Geseilschaft zur Forderung der Angewandten Forschung E.V., Munchen (DE)

(21) Appl. No.: 11/813,957

(22) PCT Filed: Jan. 9, 2006
(86) PCT No.: PCT/EP2006/000105

§ 371 Date: Jun. 16, 2008

(30) Foreign Application Priority Data
Jan. 18, 2005 (DE) 10 2005 002 343.6

(51) Int. Cl. C12N 1/02; C12N 7/02

(52) U.S. Cl. 435/239; 435/261

(57) ABSTRACT

The invention relates to a method for the specific or non-specific separation of cells and/or viruses from liquid media which is based on adsorption of the species on a correspondingly functionalised carrier material. The method according to the invention is used for separation and also isolation of any species of cells and viruses.

The invention relates to a method for the specific or non-specific separation of cells and/or viruses from liquid media which is based on adsorption of the species on a correspondingly functionalised carrier material. The method according to the invention is used for separation and also isolation of any species of cells and viruses.

The isolation and concentration of microorganisms is a large problem in microbial diagnostics. Time-consuming filtration methods (WO 01/52968) or centrifugation technologies (JP 3270701) are available here. However these are unsuccessful if the solutions to be tested are viscous or contain solid bodies. Other methods are based on immunocapturing, solid bodies which are coated with antibodies being used to collect the cells. The solid bodies can be magnetic particles (e.g. CA 1127571, AU 533747). These techniques are very expensive due to the use of antibodies. Likewise the binding of the microorganisms is greatly dependent upon external influences, such as e.g. upon temperature, flow rate and the consequently occurring shear forces.

When cleaning viruses, the application of high pressure liquid chromatography with ion exchange resins is described (WO 00/40702).

The analysis of cells, in particular animal cells, is implemented nowadays by means of flow cytometry (U.S. Pat. No. 6,793,642, US 2004189977, DE 199 45 553). The cells are thereby isolated in a capillary and sorted on the basis of a signal (e.g. light scattering, fluorescence or the like). This is a lengthy and not very selective process.

Starting herefrom, it was the object of the present invention to provide a method which enables separation and isolation of cells in a simple manner and hence is economical.

This object is achieved by the method having the features of claim 1. In claim 23, the use of the method according to the invention is described. The further dependent claims reveal advantageous developments.

According to the invention, a method for the specific or non-specific separation of cells and/or viruses from liquid media is provided, in which method
a) a carrier material is chemically functionalised on the surface in order to enable adsorption of the cells and/or viruses,
b) the carrier material is brought in contact with the liquid medium and the cells and/or viruses are adsorbed on the carrier material and
c) the carrier material laden with the cells and/or viruses is isolated from the liquid medium.

Numerous microorganisms display a fixed or reversible binding to ionic surfaces. The binding is thereby dependent upon the respective microorganism species. These interactions are however not equally strong but are different from species to species. As a result of this qualitatively and quantitatively different interaction, separation and isolation of different cells is possible, e.g. the separation of different bacterial species.

The interaction of cells and/or viruses with the surfaces of the carrier structure can be influenced by chemical functionalisation of the surface, the interaction between the cells and the carrier material being modulatable in addition by the choice of buffer via e.g. the ion concentration and the pH value. The binding can thereby be adjusted to be very strong and not detachable. On the other hand, it is likewise possible that the binding is reversible.

The carrier material is preferably selected from the group consisting of polymers, ceramics and/or glasses. The carrier material can thereby be used in the form of a powder, a granulate, a film, a membrane, a sintered body or a foam.

The surfaces of the carrier material need not necessarily be functionalised by chemical reaction. It is also possible to use the non-functionalised carrier material provided that sufficient adsorption of the cells and/or viruses is made possible in this way.

With respect to functionalisation of the surfaces of the carrier material, there are no further restrictions apart from with respect to suitability for adsorption of the cells and/or viruses or microorganisms.

Preferably, the carrier material can be functionalised in a hydrophilic but also hydrophobic manner. It is thereby possible that direct bonding of the functional groups to the carrier material is effected. A further variant provides that the functional groups are bonded to the carrier material via a spacer.

Preferably the adsorption forces which act between carrier material and cells, viruses or microorganisms concern chemisorptive interactions.

A preferred area of chemisorption is thereby based on ionic interactions. In this case, the functional groups are selected from the group consisting of ammonium, carboxyl, carboxylate, sulphonic acid, sulphonate, phosphate groups and proteins.

Another preferred variant provides that the chemisorption is based on a covalent interaction. In this case, the functional groups are selected from the group consisting of active esters, acid amides, epoxides, halogenides, acid halides and C—C multiple bonds.

With respect to the loading of the functionalised carrier material, adaptation is likewise effected with respect to the species to be separated or isolated. Hence the carrier material can have a neutral, cationic or anionic character.

The cells to be isolated are preferably selected from the group of prokaryotic and eukaryotic cells or from the group of archaebacteria. For particular preference, the cells are selected from the group consisting of bacteria, fungi, algae, spores, plant cells, animal cells, cells from cell cultures and genetically modified cells.

The conduct of the method with respect to bringing cells and/or viruses in contact with the carrier material is not designed to be restrictive and comprises all the methods known from prior art for this purpose. A variant provides for example that the carrier material is dispersed in the liquid medium. Another preferred variant provides that the carrier material is subjected to a flow of liquid medium. A third preferred variant provides that the carrier material is present in the form of a porous body, through the pores of which the liquid medium can then flow. All these variants enable adsorption of the species to be isolated provided that the interactions between carrier material and cells or viruses are strong enough.

Preferably the carrier material can be activated in addition before the functionalisation. The activation can thereby be effected chemically. A preferred variant is oxidative activation with oxidant, such as e.g. H₂O₂, peroxides, chromosulphuric acid, iodine compounds and bromine compounds. Likewise photochemical activation by means of UV light or even microwave radiation is preferred.

The chemical functionalisation is then effected by correspondingly further reactions which can be conducted also in a multistage manner with gaseous reagents, e.g. diamine, or else with liquids, e.g. solutions of polyethylene imine or polyethylene glycol.

Another variant of the activation concerns physical activation, e.g. by means of a plasma, i.e. an electrical discharge.

The isolation of the carrier material from the liquid medium is not subject to any restriction so that all the variants known from prior art can also be used here. Preferably there are included in these filtration, sedimentation or centrifugation.

A further important variant of the method according to the invention provides that at least a part of the cells and/or viruses can be recovered by elution of the laden carrier material with a liquid. Hence this hereby concerns isolation of the individual microorganism species and not solely quantitative separation.

The recovered cells and/or viruses can be preferably further treated, subsequently analysed for particular preference. This enables not only quantitative isolation of cells and/or viruses from liquids but also simultaneously a qualitative determination about which types of cells and/or viruses have been contained in the corresponding liquid.

In the case of reversible binding of cells and/or viruses to the carrier material, binding-kinetic differences of different cells to the given surface can be used for the separation. Some materials can thereby be used directly, whereas others require surface functionalisation. The elution is effected by a buffer with a corresponding ion concentration and correspondingly adjusted pH value. The individual species in this case display a different retention behaviour relative to the carrier material.

The method according to the invention is used for isolation of cells and/or viruses from liquids. There are included in these in particular, water, liquid foods, liquids from foods, body fluids or excretions. A further use concerns the separation of individual species of cells and/or viruses.

The subject according to the invention is intended to be explained in more detail with reference to the subsequent Figures and examples without said subject being intended to be restricted to the embodiment scope shown here.

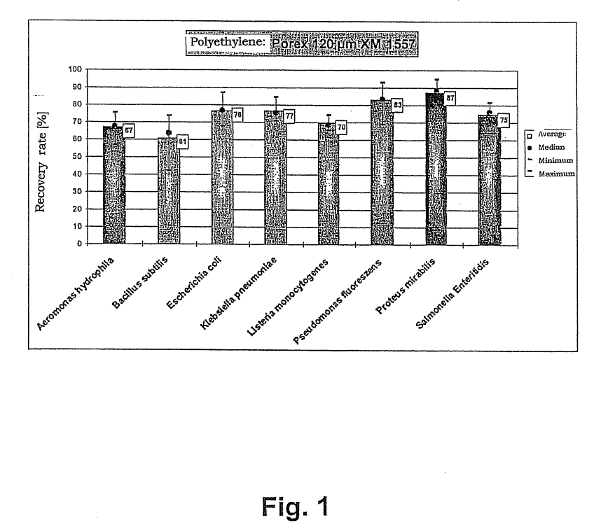

FIG. 1 shows a diagram which shows the recovery rate of specific types of bacteria in a liquid medium in which the method according to the invention has been implemented with an untreated carrier material made of polyethylene.

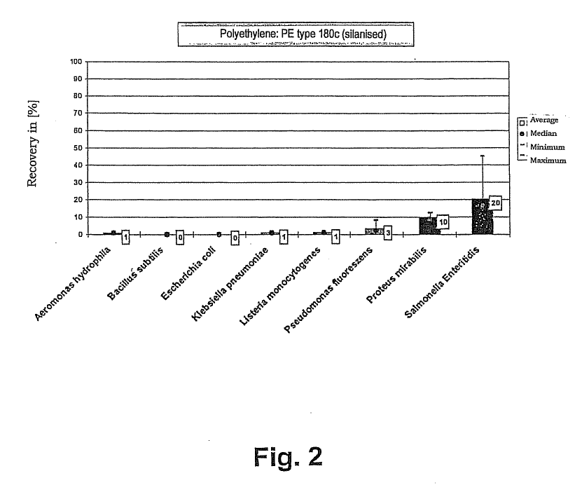

FIG. 2 shows a diagram which represents the recovery rate in a liquid medium for which the method according to the invention has been implemented with a chemically functionalised carrier material (aminated).

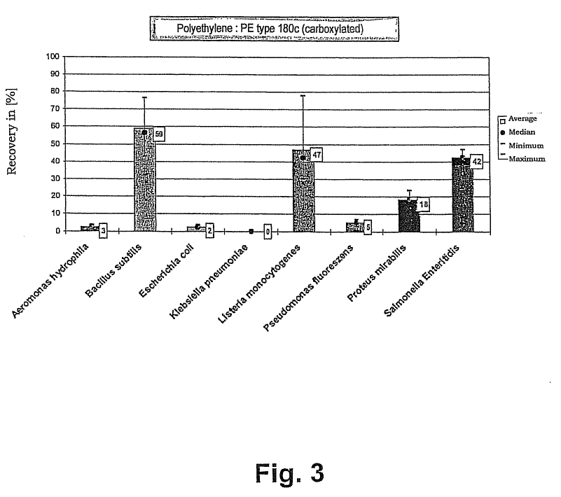

FIG. 3 shows a diagram which represents the recovery rate in a liquid medium for which the method according to the invention has been implemented with a chemically functionalised carrier material (carboxylated).

### EXAMPLE 1

Suspensions of 1.6 10⁶ KbE/g are added at a rate of 1 ml/min over cylindrical moulded articles made of sintered polyethylene powder with a 5 mm diameter and 5 mm height (without further functionalisation). The count of cells in the eluate produced the values represented in FIG. 1.

### EXAMPLE 2

Cylindrical moulded articles made of sintered polyethylene powder with a 5 mm diameter and 5 mm height are oxidised in a low pressure plasma on the surfaces and subsequently converted with aminopropyltriethoxysilane. Suspensions of 1.6 10⁶ KbE/g are added at a rate of 1 ml/min over these functionalised sintered bodies. The count of cells in the eluate produced the values represented in FIG. 2.

### EXAMPLE 3

Cylindrical moulded articles made of sintered polyethylene powder with a 5 mm diameter and 5 mm height are oxidised in a low pressure plasma on the surfaces and subsequently converted with aminopropyltriethoxysilane. Carboxyl groups are bonded to the amino groups via the reaction with succinic anhydride. Suspensions of 1.6 10⁶ KbE/g are added at a rate of 1 ml/min over these functionalised sintered bodies. The Gram-positive cells of Bacillus subtilis, Listeria monocytogenes and Salmonella enteritidis are concentrated in the eluate. The count of cells in the eluate produced the values represented in FIG. 3.

### EXAMPLE 4

A polypropylene powder is provided on the surface with a covalently bonded polyethylene imine. 0.1 g of the powder is dispersed in 11 of a suspension of 10⁴ cells per ml of E. coli. After 1 hour, the powder is filtered off bonded to the surface with all the previously dispersed cells. The cells concentrated on the powder surface are now subjected to molecular-biological analysis (e.g. with PCR).

### EXAMPLE 5

A porous body of 50 mm length and a diameter of 5 mm is fitted securely into a pressure-proof cartridge and introduced into a through-flow unit. Subsequently a sample of bacteria is applied at one end (“entry end”). This is subsequently directed over the polymer sintered body with an aqueous solution, the ion concentration and pH of which is variable. The cell material thereby interacts with the polymer sintered body and is retarded in different ways. As a result, mixtures of different bacteria can be separated and detected at the other end of the polymer sintered body (“exit end”) with a suitable detector (light scattering, UV, conductivity, fluorescence etc.). Individual bacteria fractions can be collected and, following a subsequent identification, can be identified and characterised by immunological and molecular-biological methods (e.g. PCR).

1. A method for the specific or non specific separation of cells and/or viruses from a liquid medium in which method
a) a carrier material is chemically functionalised on the surface of the carrier material in order to enable adsorption of the cells and or viruses;
b) the carrier material is brought in contact with the liquid medium and the cells and/or viruses are adsorbed on the carrier material; and
c) the carrier material laden with the cells and/or viruses is isolated from the liquid medium.

2. The method according to claim 1,
wherein the carrier material is selected from the group consisting of polymers, ceramics and glasses.

3. The method according to claim 1,
wherein the carrier material is present in the form of a powder, a granulate, a film, a membrane, a sintered body or a foam.

4. The method according to claim 1
wherein the carrier material is chemically functionalised in a hydrophilic manner.

5. The method according to claim 1,
wherein the carrier material is chemically functionalised in a hydrophobic manner.

6. The method according to claim 1
wherein the functional groups resulting from the chemical functionalisation are bonded directly to the carrier material.

7. The method according to claim 1
wherein the functional groups resulting from the chemical functionalisation are bonded to the carrier material via a spacer.

8. The method according to claim 1,
wherein the adsorption involves chemisorption.

9. The method according to claim 8,
wherein the chemisorption is based on an ionic interaction and the functional groups are selected from the group consisting of ammonium, carboxyl, carboxylate, sulphonic acid, sulphonate, phosphate groups and proteins.

10. The method according to claim 8,
wherein the chemisorption is based on a covalent interaction and the functional groups are selected from the group consisting of active esters, acid amides, epoxides, halogenides, acid halides and C—C multiple bonds.

11. The method according to claim 1,
wherein the funtionalised carrier material has a neutral, cationic or anionic character.

12. The method according to claim 1,
wherein the cells are selected from prokaryotic and eukaryotic cells and archaebacteria.

13. The method according to claim 12,
wherein the cells are selected from the group consisting of bacteria, fingi, algae, spores, plant cells, animal cells, cells from cell cultures and genetically modified cells.

14. The method according to claim 1,
wherein the carrier material is dispersed in the liquid medium.

15. The method according to claim 1,
wherein the carrier material is subjected to a flow of the liquid medium.

16. The method according to claim 1,
wherein the carrier material is present in the form of a porous body and the liquid medium flows through the pores thereof.

17. The method according to claim 1,
wherein the carrier material is activated before the functionalisation.

18. The method according to claim 17,
wherein the activation is effected chemically or physically.

19. The method according to claim 1,
wherein the isolation of the carrier material from the liquid medium is effected by means of filtration, sedimentation or centrifugation.

20. The method according to claim 1,
wherein at least some of the cells and/or viruses are recovered by elution of the laden carrier material with a liquid.

21. The method according to claim 20,
wherein the recovered cells and/or viruses are further treated.

22. The method according to claim 1, wherein the liquid medium is selected from the group consisting of water, liquid foods, liquids from foods, and body fluids or excretions.

23. The method according to claim 18 which involves separation of individual species of cells and/or viruses.
